(19) United States
(12) Patent Application Publication
(10) Pub. No.: US 2002/0037297 A1
(43) Pub. Date: Mar. 28, 2002

(54) Process for the topical treatment of rhinitis, conjunctivitis cold, and cold-like and flu symptoms

(76) Inventors: Maria Del Carmen Diez Crespo, Madrid (ES); Istvan Szelenyi, Schwaig (DE); Reinhard Muckenschnabel, Frankfurt (DE); Roberto Mainardi, Sau Paulo (BR)

Correspondence Address: GABRIEL P. KATONA L.L.P., 708 THIRD AVENUE, 14 FLOOR`, NEW YORK, NY, 10017, US

(21) Appl. No.: 09/737,252

(22) Filed: Dec. 14, 2000

Related U.S. Application Data
(63) Continuation of application No. 09/156,443, filed on Sep. 18, 1998, now abandoned.

(30) Foreign Application Priority Data
Sep. 22, 1997 (DE) 97116494.2

(51) Int. Cl. A61K 9/00

(52) U.S. Cl. 424/400

(57) ABSTRACT

A pharmaceutical composition of topically effective amounts of (i) a non-sedating antihistamine or a pharmaceutically acceptable salt thereof, together with (ii) an α-adrenergic agonist or a pharmaceutically acceptable salt thereof, and a process for the treatment of or prophylaxis against allergic rhinitis, vasomotoric rhinitis, conjunctivitis, cold, cold-like and/or flu symptoms, by topically administering the composition to mucous tissues of a patient in need therefor.

### FIELD OF THE INVENTION

The present invention relates generally to novel pharmaceutical compositions for the topical treatment of rhinitis, conjunctivitis cold, and cold-like and flu symptoms.

### BACKGROUND

In industrialized countries, more than 10-15% of the population suffer from allergic rhinitis and/or conjunctivitis. Allergic rhinitis and/or conjunctivitis are type I allergic responses that are mediated by IgE antibodies. As a part of an allergic response to antigen, reaginic antibodies (IgE) are generated and bound to the surface of mast cells and basophils via high affinity Fc receptors (FceRI) that are specific for IgE. Antigen cross-linking the IgE-molecules leads to cellular responses involving release of performed mediators (e.g. histamine), lipid mediator formation and release, and cytokine generation. Mast cells with their mediators can be regarded as central to the initiation and mediation of the early phase of allergic inflammation.

Symptoms of rhinitis are sneezing, itching (nasal irritation), rhinorrhea (nasal secretion) and nasal blockage (congestion). Nasal blockage is the result of the pooling of blood in the capacitance vessels of the mucosa, and to some degree the result of tissue oedema. Patients with allergic conjunctivitis show similar symptoms.

Itching, eye rubbing and tearing are very common, and cause much distress. Conjunctival oedema and hyperemia cause the bulbar surface to take on a glassy appearance, with dilated blood vessels.

The common cold is usually not a serious illness but it is highly prevalent, discomforting, and annoying infliction. The term common cold is applied to minor upper respiratory illnesses caused by a variety of different respiratory viruses, in where rhinoviruses are the major known cause of common cold. Symptoms such as nasal discharge, nasal blockage, and sneezing usually commence on the first day of illness and progress to maximum severity by the 2nd and 3rd day. Along with nasal symptoms may come other symptoms such as cough, burning of the eyes, headache. Fever can also occur.

Antihistamines are generally used in the symptomatic treatment of these disorders. They are effective in reducing itching, sneezing and watery secretion. Based on the ICAM-1(rhinovirus binding site) down-regulating effect of azelastine, it may be particularly useful in the treatment of common cold/flu. Antihistamines are, in general, less effective in the reduction of nasal congestion (blockage). To achieve a further reduction of nasal congestion and ocular oedema, α-adrenergic agonists are often used either alone or in combination with antihistamines.

Non-sedating antihistamines especially such administered topically represent a new generation of histamine H₁ receptor antagonists. They possess strong antagonistic activity at histamine H₁ receptors without causing sedation. For example, azelastine, a phthalazinone derivative, belongs to this so-called second-generation non-sedating antihistamines. It is characterized by a long-lasting antiallergic activity and shows a broad spectrum of pharmacological activities including not only antagonism of histamine H,-receptors but also inhibition of histamine release following antigen and non-antigen stimuli (Chand, N. et al. Inhibition of allergic and nonallergic leukotriene C4-formation and histamine secretion by azelastine: Implication for its mechanism of action. Int. Arch. Allergy Appl. Immunol. 90:67-70, 1989). Recently, it has also been demonstrated that topically administered azelastine down-regulates ICAM-1 (intercellular adhesion molecule-1), the binding site for rhinoviruses (Ciprandi, M. D. et al. Topical azelastine reduces eosinophil activation and intracellular adhesion molecule-1 expression on nasal cells: An antiallergic activity. J. Allergy Clin. Immunol. 98:1088-1096, 1996). Azelastine is free of cardiovascular side effects. Because the drug is administered topically, the plasma levels following topical azelastine application are extremely low, even if it is overdosaged. By contrast, oral formulations containing antihistamines of the second generation such as terfenadine, astemizole, loratadine may cause cardiovascular side effects (e.g. tachyarrhythmias) when the recommended dosage is not kept.

As disclosed in WO 94/08551, with regard to allergic diseases and the common cold, either topical or oral α-adrenergic agonists are used. Oral decongestants (e.g. pseudoephedrine, phenylephrine, phenylpropanolamine as disclosed in, for example, WO 92/04021, WO 92/04022, WO 94/08550, WO 94/14449 and WO 95/23591) carry the risk of inducing systemic adverse effects such as tachycardia, increased blood pressure, and CNS stimulation (e.g. insomnia). Topical α-adrenergic agonists such as epinephrine, fenoxazoline, indanazoline, naphazoline, oxedrine, oxymetazoline, phenylephrine, tefazoline, tetryzoline, tramazoline, tymazoline, xylometazoline are used as local decongestants in patients with allergic or vasomotor rhinitis, conjunctivitis or with upper respiratory infections (e.g. common cold, flu). α-adrenergic drugs probably decrease resistance to airflow by decreasing the volume of the nasal mucosa. This may occur by activation of α-adrenergic receptors in venous capacitance vessels in nasal tissues. Topical decongestants are particularly useful because of their more selective site of action. Although their topical administration is associated with few systemic adversed effects, prolonged use may result in rebound congestion and worsening of symptoms. Therefore, the use of formulations containing a nasal vasoconstrictor compound is not recommended longer than 7-10 days. In addition, the recommended daily dosage should not be exceeded.

Combinations containing antihistamines and α-adrenergic agonists are widely used orally both for the treatment of allergic rhinitis and common cold as disclosed in WO 88/09656, WO 94/14476, WO 94/25009 and WO 95/07103. It has been demonstrated that patients suffering from common cold or allergic rhinitis have benefited from oral antihistamine+decongestant therapy (Anonymous: The management of hay fever. Drug Ther. Bull. 23:25-27, 1985; Berkowitz, R. B. et al. The effectiveness of the nonsedating antihistamine loratadine plus pseudoephedrine in the symptomatic management of the common cold. Ann. Allergy 63:336-339, 1989). Topical α-adrenergic agonists in combination with antihistamines are administered in which sedating antihistamines (e.g. antazoline) are used. Topical decongestants (α-adrenergic agonists) provide quick relief of nasal or ocular oedema. However, topical formulations containing second generation antihistamines and a-adrenergic agonists are not available and are not known. In considering the advisability of topical vs. systemic applications, the common wisdom has been that “[A]ntihistamines should never be applied locally.” [R. Lancaster, Topical or Systemic Therapy, Prescriber's Journal, 23/2, 1983, pp. 47-53]; “[T]he benefit of administering antihistamines intranasally is doubtful . . . [due to report of] the occurrence of severe irritative thinitis and allergic reactions . . . [which] indicates that the intranasal application of antihistamines should be discouraged.” [H. J. M. van de Donk et al.: The Effects of Drugs . . . , Intern. Jnal. of Pharmaceut., 12 (1982) pp. 77-85]; and that “[I]t is generally considered that local application of antihistamines carries an unacceptably high risk of skin sensitization.” [Martindale The Extra Pharmacopoeia, The Pharmaceutical Press, London 1989 p. 443].

Formulations for the treatment of cold, cold-like and/or flu symptoms often contain antihistamines and decongestants, but only oral combination preparations are being used.

### SUMMARY OF THE INVENTION

It is an object of the present invention to provide novel pharmaceutical compositions for topical application based on the surprising discovery that the conventional concerns about topical application do not apply in the case of second generation, nonsedating antihistamines, especially when combined with an α-adrenergic agonist, because these compositions do not manifest the strong reactions that were of so much concern in the case of conventional antihistamines. Accordingly, the compositions of the present invention comprise a pharmacologically effective amount of a non-sedating antihistamine, suitably at least one acrivastine, antazoline, astemizole, azelastine, cetirizine, ebastine, efletirizine, epinastine, fexofenadine, loratidine, levocabastine, mizolastine, oxatomide, setastine, temelastine and terfenadine, in combination with an α-adrenergic agonist, suitably one or more of epinephrine, fenoxazoline, indanazoline, naphazoline, oxedrine, oxymetazoline, phenylephrine, tefazoline, tetryzoline, tramazoline, tymazoline and xylometazoline, and optionally a pharmacologically acceptable carrier and/or diluent or any auxiliary substance therefor.

It is a further object of the present invention to provide a method for the prophylaxis and treatment of allergic and/or vasomotoric rhinitis, conjunctivitis, cold, cold-like and/or flu symptoms in a mammalian host in need of such treatment by topically administering a pharmacologically effective amount of the composition of the present invention.

It is yet another object of the present invention to provide suitable dosage unit forms of the composition of the present invention for convenient topical administration, such as in the form of spray or drops.

### DETAILED DESCRIPTION

The antiallergic component in the pharmaceutical combination of the present invention includes a non-sedating antihistamine applied topically such as suitably acrivastine, antazoline, astemizole, azelastine cetirizine, ebastine, efletirizine, epinastine, fexofenadine, loratidine, levocabastine, mizolastine, oxatomide, setastine, temelastine or terfenadine or a pharmacologically acceptable salt thereof. Azelastine, a particularly suitable agent is a second generation histamine H₁-receptor antagonist without sedating effect.

The concentration of the antihistaminic component of the composition of the present invention is suitably from about 0.001% to about 0.5% wt., most suitably of from about 0.05% to about 0.1% wt.

In addition to the antihistaminic component the composition contains a topical decongestant, suitably epinephrine, fenoxazoline, indanazoline, naphazoline, oxedrine, oxymetazoline, phenylephrine, tefazoline, tetryzoline, tramazoline, tymazoline or xylometazoline, or a pharmacologically acceptable salt thereof. The concentration of α-adrenergic agonists in the combination is suitably from about 0.001% to about 0.2% wt., most suitably of from about 0.05% to about 0.1% wt. In the special case of phenylephrine the concentration thereof is suitably of from about 0.01% to about 15% wt., most suitably from about 0.1% to about 2% wt. The active ingredients are administered topically as a mixture containing pharmaceutical diluents, excipients or a carrier consistently with conventional pharmaceutical practices.

The pharmaceutical composition of the present invention is suitably administered for nasal application as 1 puff per nostril twice daily with a maximum daily dose of about 3 puffs per nostril; and as eye drops 1 drop in each eye twice daily with a maximum daily dose of about 3-6 drops per eye.

In addition to the active ingredients the compositions of the present invention can further comprise one or more of various ingredients such as antimicrobial preservatives, tonicity agents, thickening agents, excipients for pH-adjustment and buffers.

For example antimicrobial preservatives can include benzalkonium chloride, chlorobutanol, thiomersal, methylparaben, propylparaben, sorbic acid, edetate disodium, phenylethanol, chlorhexidine HCl, chlorhexidine acetate, chlorhexidine digluconate, cetylpyridinium chloride or bromide, chlorocresol, phenylmercuric acetate, phenylmercuric nitrate, phenylmercuric borate, and phenoxyethanol.

As a preservative suitably a combination of disodium edetate and benzalkonium chloride is used. Disodium edetate is suitably used in concentrations of from about 0.05 to about 0.1% and benzalkonium chloride in concentrations of from about 0.005 to about 0.05%. Suitable excipients which can be used to adjust tonicity or osmolality can include sodium chloride, potassium chloride, mannitol, glucose, sorbitol, glycerol, and propylene glycol. In general these agents are used in concentrations of from about 0.1 to about 10%.

The compositions can suitably contain thickening agents to increase the viscosity and prolong contact of the drug with the tissue. Thickening agents can suitably be methylcellulose, hydroxypropyl methylcellulose, hydroxyethylcellulose, sodium carboxymethylcellulose, polyvinyl alcohol, polyvinyl pyrrolidone, polyacrylates, polyacrylamide, dextran, gellan gum (Gelrite), poloxamere, and cellulose acetate phthalate.

The compositions of the present invention can suitably also include pharmaceutically acceptable buffers sufficient to adjust and maintain the pH in the range of from about 4 to about 8, most suitably from about 5.5 to about 7.5.

Suitable buffers include citrate, phosphate, tromethamine, glycine, borate, and acetate. These buffers can be built from substances like citric acid, monosodium phosphate, disodium phosphate, glycine, boric acid, sodium tetraborate, acetic acid, and sodium acetate. Also other excipients can be used for pH-adjustment such as hydrochloric acid and sodium hydroxide.

Further details of this invention are given in the following examples.

### EXAMPLE 1

Nasal spray or nasal drops containing azelastine HCl (0.1%) and oxymetazoline HCI (0.05%)

| | |
|---|---|
| azelastine HCl | 0.01000 g |
| oxymetazoline HCl | 0.00500 g |
| hydroxypropyl methylcellulose | 0.01000 g |
| disodium edetate | 0.00500 g |
| benzalkonium chloride | 0.00125 g |
| citric acid anh. | 0.00438 g |
| disodium phosphate dodecahydrate | 0.04655 g |
| sorbitol soln. (70%) | 0.60000 g |
| purified water | q.s. 10.0 ml |

### EXAMPLE 2

Eye drops containing azelastine HCI (0.05%) and tetryzoline HCI (0.05%)

| | |
|---|---|
| azelastine HCl | 0.00500 g |
| tetryzoline HCl | 0.00500 g |
| hydroxypropyl methylcellulose | 0.01000 g |
| disodium edetate | 0.00500 g |
| benzalkonium chloride | 0.00125 g |
| sodium hydroxide | q.s. pH 6.0 |
| sorbitol soln. (70%) | 0.66666 g |
| water for injections | q.s. 10.0 ml |

### EXAMPLE 3

For nasal spray or nasal drops see Example 1, but with 0.1% xylometazoline HCl instead of 0.05% oxymetazoline HCI

### EXAMPLE 4

For eye drops see Example 2, but with 0.1% naphazoline HCI instead of 0.05% tetryzoline HCl

### EXAMPLE 5

For nasal spray or nasal drops see Example 1, but with 0.05% naphazoline HCI instead of 0.05% oxymetazoline HCI

### EXAMPLE 6

For nasal spray or nasal drops see Example 1, but with 0.1264% tramazoline HCl instead of 0.05% oxymetazoline HCl

### EXAMPLE 7

For eye drops see Example 2, but with 0.0632% tramazoline HCl instead of 0.05% tetryzoline HCI

### Preparation of Eye Drops of Examples 2, 4 and 7

Prepare 45.0 kg of water for injections in a suitable container and dissolve therein while stirring the active principles, disodium edetate, benzalkonium chloride, hydroxypropyl methylcellulose and sorbitol solution. Fill up the solution with water for injection to 49.5 l. Adjust the pH of the solution with sodium hydroxide solution 1N to pH 6. Fill up the solution with water for injections to get 50.0 l and stir. Filter the solution under sterile conditions through a membrane filter of a pore size of 0.2 μm, and fill the solution aseptically into sterilized bottles.

### Preparation of Nasal Sprays or Nasal Drops For Examples 1, 3, 5 and 6

Prepare 96.5 kg of purified water in a suitable container and dissolve therein while stirring the active principles, disodium edetate, sorbitol solution, benzalkonium chloride, disodium phosphate dodecahydrate, citric acid anhydrous and hydroxypropyl methylcellulose. Fill up the solution to 100 l and stir. Filter the solution through a membrane filter of pore size 0.2 μm and fill it into bottles.

We claim:

1. A pharmaceutical composition which comprises topically effective amounts of (i) a non-sedating antihistamine or a pharmaceutically acceptable salt thereof, together with (ii) an α-adrenergic agonist or a pharmaceutically acceptable salt thereof.

2. The pharmaceutical composition of claim 1, wherein said non-sedating antihistamines is at least one of acrivastine, antazoline, astemizole, azelastine, cetirizine, ebastine, efletirizine, epinastine, fexofenadine, loratidine, levocabastine, mizolastine, oxatomide, setastine, temelastine, and terfenadine.

3. The pharmaceutical composition of claim 1, wherein said α-adrenergic agonist is at least one of epinephrine, fenoxazoline, indanazoline, naphazoline, oxedrine, oxymetazoline, phenylephrine, tefazoline, tetryzoline, tramazoline, tymazoline, and xylometazoline.

4. The pharmaceutical composition of either of claim 1 and 2, wherein said non-sedating antihistamine is present at a concentration of from about 0.001% to about 0.5%.

5. The pharmaceutical composition of either one of claims 1 and 2, wherein said non-sedating antihistamine is present at a concentration of from about 0.05% to about 0.1%.

6. The pharmaceutical composition of either one of claims 1 and 3, wherein said α-adrenergic agonist, except phenylephrine, is present at a concentration of from about 0.001% to about 0.2%, and when said α-adrenergic agonist is phenylephrine then at a concentration of from about 0.015% to about 15%.

7. The pharmaceutical composition of either one of claims 1 and 3 wherein said α-adrenergic agonist, except phenylephrine, is present at a concentration of from about 0.05% to about 0.1%, and when said α-adrenergic agonist is phenylephrine then at a concentration of from about 0.1% to about 2%.

10. The pharmaceutical composition of claim 1, when formulated into a topical dosage form.

11. The pharmaceutical composition of claim, 11, wherein said topical dosage form is a nasal spray, nose drop, or eye drop.

12. The pharmaceutical composition of claims 1 to 10 in topical dosage spray form.

13. A process for the topical treatment of or prophylaxis against allergic rhinitis, vasomotoric rhinitis, conjunctivitis, cold, cold-like and/or flu symptoms, which comprises topically administering to mucous tissues of a patient in need therefor a therapeutically effective amount of the composition of claim 1.
